(19) United States
(12) Patent Application Publication
(10) Pub. No.: US 2008/0031863 A1
(43) Pub. Date: Feb. 7, 2008

(54) Method of Preparation and Use of Fibrinolytic Enzymes in the Treatment of Disease

(75) Inventors: DeWall J. Hildreth, Green Valley, AZ (US); Tiffany Thomas, Phoenix, AZ (US)

Correspondence Address: BROWN, RUDNICK, BERLACK & ISRAELS, LLP., BOX IP, 18TH FLOOR, ONE FINANCIAL CENTER, BOSTON, MA, 02111, US

(73) Assignee: HEALTH ENHANCEMENT PRODUCTS, INC., Scottsdale, AZ (US)

(21) Appl. No.: 11/587,266

(22) PCT Filed: Apr. 20, 2005
(86) PCT No.: PCT/US05/13375

§ 371 Date: Aug. 14, 2007

Related U.S. Application Data
(60) Provisional application No. 60/565,011, filed on Apr. 23, 2004.

(51) Int. Cl. A61K 38/54; A61K 38/55; A61P 17/10; A61P 3/00; A61P 3/10; A61P 31/18; A61P 35/00; A61P 9/00; C12P 21/00

(52) U.S. Cl. 424/94.2; 435/70.1

(57) ABSTRACT

This invention relates generally to a method of preparation of a phyto-percolate having enzymatic, specifically fibrinolytic, activity in the body of mammals, including humans. The phyto-percolate is derived from fresh water mixture including algae that produces an enzyme having proteolytic activity. The invention further relates to the use off the phyto-percolate in a variety of disease states.

### FIELD OF INVENTION

This invention relates generally to a method of preparation of a phyto-percolate believed to have enzymatic activity. The phyto-percolate is a proteolytic enzyme complex derived from fresh water algae that expresses plasmin-like activity.

### BACKGROUND OF INVENTION

Enzymes have a very important use within biochemical cycles in the human body. The majority of acute and chronic diseases create an inflammatory process that results in the destruction of surrounding tissue. This tissue debris becomes toxic and further hinders the processes of detoxification, elimination and defense by way of free radical oxidation. Proteolytic enzymes are responsible for the body's detoxification processes. As humans age and chronic disease processes progress, a deficiency of the proteolytic enzymes that carry out the body's waste detoxification processes may be experienced. This enzymatic deficiency aids in the production of a chronic hyper-inflammatory state, and the disease process becomes much more complex.

Enzymes are the catalysts that control and direct all metabolic processes. Without adequate enzymes in the body, chaos reigns and the immune system and other metabolic processes become less efficient, making tissue repair slow and poorly replicated. Proteolytic enzymes, or proteases, are enzymes capable of breaking down proteins by cleaving peptide bonds. They are produced and utilized by every living organism on Earth for protection, nutrient breakdown and assimilation, and waste removal. Many degenerative diseases stem from proteolytic enzyme deficiencies, leading to the inadequate removal of carcinogenic wastes from the body.

It is believed that the immune system, which helps protect us from diseases including cancer, cardiovascular disease, and other immune deficient or deregulated disorders, can become ineffective because of advanced disease state or age. Immune deficiency caused by disease state or advancing age can impair benefits received from the use of therapeutic drugs that may be taken for the treatment of these various disorders. Therapeutic drugs may lose their effectiveness in a compromised immune system as a disease state progresses due to metabolic dysfunction or poor therapeutic drug assimilation.

With advancing age, humans experience an increasing accumulation of environmental influences that are believed to have toxic effects on the human body. An observed effect associated with aging is a less accurate tissue repair process, possibly an expression of DNA mutations caused by environmental factors, Because of these alterations, foreign antigens in the way of microbes, and environmental toxins such as radiation and chemical compounds through foods, water, and air, are allowed to increasingly invade the human organism. These environmental toxins are introduced primarily through the mucous membranes of the intestinal tract, upper respiratory tracts and lungs.

Human genes, which are made up of double-strands of DNA, are the directors of tissue repair. It is believed that through advancing age and contact with the surrounding destructive elements, the expression of such DNA may become less and less accurate because of replication errors and mutations, thus creating very different functional end products of repair when compared to a younger individual.

Impaired immune protection and regulation, it is believed, allows an increasing amount of toxic environmental components to invade the cells of our bodies. These toxic components express destructive patterns of oxidation by way of free radical activity, thus rendering important metabolic processes to function inadequately. Because of biochemical cellular destruction, dead, fractionated cellular components are created, adding to the toxic manifestations. White cells, which are an important part of the immune system, congregate at the sites of tissue destruction in an effort to slow the process down. A chemical reaction that takes place at the site causes inflammation that further increases the destructive pattern. This pattern of tissue destruction, secondary to foreign antigen invasion and the associated white cell activity, can create an ongoing autoimmune hyperactive inflammatory state and an increasing amount of toxic tissue destruction and debris. Because of the increased inefficiency of tissue repair and the ever presence of surrounding environmental influences, human metabolic processes become less and less efficient with age.

The inner lining of the blood vessels, particularly the arteries, can be affected by this destructive pattern. Because many environmental contaminants are introduced into human bodies through the intestinal tract and lungs, they spread through the body by way of the vascular bed, thus coming first in contact with the inner lining of the blood vessels. This ongoing contact in the inner lining of the arteries with toxic free radicals results in the destructive oxidative process. This maintains an ongoing inflammatory state that includes cell break down and scar tissue formation in the form of sclerotic plaques. These plaques are made up of fibrous tissue, cholesterol, calcium deposits and necrotic tissue (broken down cellular components). Increasing arterial restriction and blood thickening due to pathological fibrin diminishes blood flow and alters oxygen and nutrient distribution to vital organs. This gradually increasing cellular starvation affects the functions of the brain, heart, kidneys, muscles, joints and other vital systems.

It is believed that accelerated DNA mutations and errors in replication, increased oxidation, inflammation, dysregulated white cell activity, and tissue destruction are the results of a gradual progression of contact with environmental forces, including pathogenic microbials. The amount of contact depends on, lifestyle and individual health care. Some illnesses either originate from excessive free radical oxidation destruction at the body's cellular level, or cause a great increase in free radical oxidation destruction. Therefore, when the body's own metabolic and healing processes are unable to cope with the excess of toxic waste products, a cycle of ongoing inflammation and disease is created that interferes with the body's normal immune activity and tissue repair. Tissue destruction also activates the body's coagulation, or blood-clotting, mechanism, generating a barrage of intra-vascular thrombi, or blood clots, and blood-thickening fibrin, that can precipitate strokes, heart attacks, pulmonary emboli, kidney damage, and phlebitis.

Oxidative free radical activity becomes rampant because of the action of the involving white cells attempting to control the initial cause of the destruction. The resulting pathological agents secondary to this influence of white cell activity create an ongoing destructive pattern upon local surrounding tissue, the endothelial cells that line the vascular bed, and the epithelial cells lining the intestinal tract. Not only is there destructive activity upon the above-mentioned tissues but also there is oxidative breakdown or pathological activation of the coagulation factors. This includes pathologically activated fibrinogen to produce a soluble fibrin that, unlike insoluble fibrin, which is an important component of the normal blood-clotting mechanism, cannot be cross-linked and is pathological, or harmful to the body. This soluble fibrin not only negatively influences general capillary circulation but also kidney filtration, oxygen exchange within the alveoli of the lungs, and oxygenation of brain tissue. It not only thickens the blood, but is in itself an oxidative free radical, and contributes to the degenerative oxidation process.

Much of the expressed symptomotology from the production of soluble fibrin is caused by gram-negative bacteria, mycoplasma and Candida albicans, which are allowed to flourish in the immune-compromised environment created by excess wastes and fibrin, and is related to the cellular destruction and by-products of ongoing free radical activity. Fibrinolytic activity, or the process of breaking down fibrin, along with the eradication of the foreign pathological agents by other therapeutic interventions, can lead to increasingly effective immune system and white cell activity, and will greatly accelerate the healing process

Most cancer processes liberate hydrogen peroxide, which acts as a free radical oxidative agent. In addition to hydrogen peroxide, the effects of cancer growth and chemotherapy produce excess soluble fibrin products as a response to these abnormal and destructive processes. The fibrin is produced as part of the body's natural reaction to tissue damage, which also occurs normally at the site of a superficial wound. However, at the site of cancer growth, fibrin coats cancer cells, tragically insulating them from destruction by the body's immune system. These coagulation mechanisms, stimulated by the oxidative damage associated with chronic illness, the damaging effects of chemotherapy, and the nature of abnormal cancer growth, all lead to further damage. Chronic illnesses such as cancer produce an acceleration of disseminated intravascular coagulation, causing not only a build-up of soluble fibrin but also of small intravascular thrombi, or clots that float around the vascular bed acting as emboli that obstruct circulation. The use of a fibrinolytic agent, along with any other therapeutic regime, will increase immune regulation and the effectiveness of white cell activity, improve capillary circulation and nutrient flow to the body's organs, aid in eliminating toxins, and enhance the benefits of other therapeutic agents. In addition, fibrinolytic agents will reduce the amount of free radical soluble fibrin that accelerates degenerative oxidation, and can increase the body's immune effectiveness in combating cancer growth.

In vivo laboratory monitoring of disease processes has supported the observations that improved cellular function and efficiency come with less oxidative, free radical activity, improved cellular nutrition, enhanced immune activity and white cell function and improved oxygenation.

### SUMMARY OF THE INVENTION

According to the invention, a method for treating or preventing a disease or disorder in a human is capable of being treated or prevented by a phyto-percolate derived from mixtures including freshwater algae. The inventive method comprises administering to a human in need of such treatment or prevention a therapeutically effective amount of phyto-percolate.

An aspect of this invention is directed to a method for treating immune system deficiency or disregulation comprising administering to a subject mammal (e.g., a female or male human, or animal) a therapeutically effective amount of a phyto-percolate.

In particular embodiments the subject of treatment has a disease or condition therapeutically treated by administering a therapeutically effective amount of a phyto-percolate. For humans, a particular dosage is between about 1 and about 8 ounces per day of the phyto-percolate. Particularly noted is a dosage of about 1 to about 4 ounces per day Another aspect of this invention is directed to a method of treating obesity comprising administering to a mammal (e.g., a female or male human) in need of such treatment a therapeutically effective amount of phyto-percolate.

Another aspect of this invention is directed to a method for treating eating and metabolic disorders, some which may lead to obesity, comprising administering to a mammal (e.g., a female or male human) a therapeutically effective amount of a phyto-percolate.

Another aspect of this invention is directed to a method for treating hyperlipidemia comprising administering to a mammal (e.g., a female or male human) a therapeutically effective amount of a phyto-percolate.

Another aspect of this invention is directed to a method for treating cellulite and fat accumulation comprising administering to a mammal (e.g., a female or male human) a therapeutically effective amount of a phyto-percolate.

Another aspect of this invention is directed to a method for treating type I and II diabetes comprising administering to a mammal (e.g., a female or male human) a therapeutically effective amount of a phyto-percolate.

Another aspect of this invention is directed to a method for treating diseases related to the heart, blood vessels or renal system comprising administering to a mammal (e.g., a female or male human) a therapeutically effective amount of a phyto-percolate.

Another aspect of this invention is directed to a method for treating a vasospasm comprising administering to a mammal (e.g., a female or male human) a therapeutically effective amount of a phyto-percolate.

Another aspect of this invention is directed to a method for treating heart failure comprising administering to a mammal (e.g., a female or male human) a therapeutically effective amount of a phyto-percolate.

Another aspect of this invention is directed to a method for treating cardiac hypertrophy comprising administering to a mammal (e.g., a female or male human) a therapeutically effective amount of a phyto-percolate.

Another aspect of this invention is directed to a method for treating dysregulated blood pressure comprising administering to a mammal (e.g., a female or male human) a therapeutically effective amount of a phyto-percolate

Another aspect of this invention is directed to a method for treating angina comprising administering to a mammal (e.g., a female or male human) a therapeutically effective amount of a phyto-percolate.

Another aspect of this invention is directed to a method for treating peripheral vascular disease comprising administering to a mammal (e.g., a female or male human) a therapeutically effective amount of a phyto-percolate.

Another aspect of this invention is directed to a method for treating cerebral diseases and diseases related to the central nervous system that are vascular in origin comprising administering to a mammal (e.g., a female or male human) a therapeutically effective amount of a phyto-percolate.

Another aspect of this invention is directed to a method for treating neuro-degeneration comprising administering to a mammal (e.g., a female or male human) a therapeutically effective amount of a phyto-percolate.

Another aspect of this invention is directed to a method for treating Alzheimer's disease comprising administering to a mammal (e.g., a female or male human) a therapeutically effective amount of a compound of a phyto-percolate.

Another aspect of this invention is directed to a method for treating depression comprising administering to a mammal (e.g., a female or male human) a therapeutically effective amount of a phyto-percolate.

Another aspect of this invention is directed to a method for treating addiction, including post-drug detoxification and/or substance abuse including nicotine, cocaine and alcohol abuse comprising administering to a mammal (e.g., a female or male human) a therapeutically effective amount of a phyto-percolate.

Another aspect of this invention is directed to a method for treating attention deficit disorder comprising administering to a mammal (e.g., a female or male human) a therapeutically effective amount of a compound of a phyto-percolate.

Another aspect of this invention is directed to a method for treating sleep disorders comprising administering to a mammal (e.g., a female or male human) a therapeutically effective amount of a phyto-percolate.

Another aspect of this invention is directed to a method for treating seasonal affective disorder comprising administering to a mammal (e.g., a female or male human) a therapeutically effective amount of a phyto-percolate.

Another aspect of this invention is directed to a method for treating environmental and food allergies comprising administering to a mammal (e.g., a female or male human) a therapeutically effective amount of a phyto-percolate.

Another aspect of this invention is directed to a method for treating conditions related to pain or nociperception comprising administering to a mammal (e.g., a female or male human) a therapeutically effective amount of a phyto-percolate.

Another aspect of this invention is directed to a method for treating migraine comprising administering to a mammal (e.g., a female or male human) a therapeutically effective amount of a compound of a phyto-percolate.

Another aspect of this invention is directed to a method for treating disorders related to disruption of circadian rhythms including jet lag comprising administering to a mammal (e.g., a female or male human) a therapeutically effective amount of a phyto-percolate.

Another aspect of this invention is directed to a method for treating diseases related to abnormal gastrointestinal motility and secretion comprising administering to a mammal (e.g., a female or male human) a therapeutically effective amount of a phyto-percolate.

Another aspect of this invention is directed to a method for treating diarrhea and/or fecal incontinence comprising administering to a mammal (e.g., a female or male human) a therapeutically effective amount of a phyto-percolate.

Another aspect of this invention is directed to a method for treating a gastric ulcer comprising administering to a mammal (e.g., a female or male human) a therapeutically effective amount of a phyto-percolate.

Another aspect of this invention is directed to a method for treating irritable bowel syndrome comprising administering to a mammal (e.g., a female or male human) a therapeutically effective amount of a phyto-percolate.

Another aspect of this invention is directed to a method for treating inflammatory bowel disease comprising administering to a mammal (e.g., a female or male human) a therapeutically effective amount of a phyto-percolate.

Another aspect of this invention is directed to a method for treating nausea comprising administering to a mammal (e.g., a female or male human) a therapeutically effective amount of a phyto-percolate.

Another aspect of this invention is directed to a method for treating sexual dysfunction comprising administering to a mammal (e.g., a female or male human) a therapeutically effective amount of a phyto-percolate.

Another aspect of this invention is directed to a method for altering fertility comprising administering to a mammal (e.g., a female or male human) a therapeutically effective amount of a phyto-percolate.

Another aspect of this invention is directed to a method for treating conditions or disorders associated with inflammation comprising administering to a mammal (e.g., a female or male human) a therapeutically effective amount of a phyto-percolate. Inflammation may be the cause of many of the above and below listed diseases such as allergies, anal fissures, angina, active ankylosing spondylitis, arterial occlusion, arteriosclerosis, atherioscleriosis, arthritis, arthorsis, asthma, auto-immune diseases, bronchitis, bronchospasm, cellulitis, cholecystitis, chronic pain, cirrhosis of the liver, colitis, conjunctivitis, contact dermatitis, C.O.P.D., cystitis, dermatitis, diabetes, diarrhea, diverticulitis, eczema, encephalitis, endocarditis, endometritis, enteritis, erythemas, glomerular disease, gout, hemorrhoids, hepatitis, elietis, leukoencephalopathy, lupus, menier's disease, meningitis, myalgia, myocarditis, myositis, myonecrosis, otitis media, pancreatitis, pandiculitis, polyateritis, proctitis, radiculoneuritis, rheumatism, rheumatoid arthritis, rhinitis, salpingitis, sinusitis, skin burns, stomatitis, strokes, tendonitis, thrombophlebitis, thrombosis, thyroiditis, tinnitus, urethritis, urticaria and vasulitis.

Another aspect of this invention is directed to a method for treating conditions or disorders associated with the immune system comprising administering to a mammal (e.g., a female or male human) a therapeutically effective amount of a phyto-percolate. Immune system deficiency may be the cause of many of the above and below listed diseases such as cancer, emphysema, encephalitis, environmental sensitivity, erysipelas, food poisoning and Reynaud's disease.

Another aspect of this invention is directed to a method for treating conditions or disorders associated with infectious disease comprising administering to a mammal (e.g., a female or male human) a therapeutically effective amount of a phyto-percolate. Infectious disease may be the cause of many of the above and below listed diseases such as pneumonia, all viruses, acariosis, acne, adenovirus, AIDS, amebiasis, anthrax, athlete's food, babesiosis, bartonellosis, Bell's palsy, botulism, candidiasis, carbuncles, Chaga's disease, chicken pox, Chlamydia, coccidiomycosis, coronavirus, cryptococcosis, cytomegalovirus, Dengue fever, echovirus, erysipelas, furuncle, gangrene, Guillan-Barre syndrome, hepatitis, impetigo, influenza, leucopenia, Lyme's disease, malaria, martolditis, measles, mumps, mycobacterium, mycosis, parasites, pediculosis, P.I.D. pyodermia, rabies, rubella, salmonella, salpingitis, septicemia, shingles, sinusitis, syphilis, tetanus, Tindi Cruzi and warts.

Another aspect of this invention is directed to a method for treating conditions or disorders associated with hormonal imbalances comprising administering to a mammal (e.g., a female or male human) a therapeutically effective amount of a phyto-percolate. Hormonal imbalances may be the cause of many of the above and below listed diseases such as acne, Addison's disease, endometritis, Grave's disease, osteoporosis, menopausal regulation and glucose regulation.

Another aspect of this invention is directed to a method for treating conditions or disorders associated with neurological deficiencies comprising administering to a mammal (e.g., a female or male human) a therapeutically effective amount of a phyto-percolate. Neurological deficiencies may be the cause of many of the above and below listed diseases such as Lou Gehrig's disease, chronic pain, Huntingdon's Chorea, multiple sclerosis, Myasthenia Gravis, Parkinson's disease, poliomyelitis, senile dementia, striatonigral degeneration, stroke, tardine dyskinesia and tinnitus

Another aspect of this invention is directed to a method for treating respiratory diseases comprising administering to a mammal (e.g., a female or male human) a therapeutically effective amount of a phyto-percolate.

Another aspect of this invention is directed to a method for treating asthma comprising administering to a mammal (e.g., a female or male human) a therapeutically effective amount of a phyto-percolate.

Another aspect of this invention is directed to a method for treating diseases related to abnormal hormone release and utilization comprising administering to a mammal (e.g., a female or male human) a therapeutically effective amount of a phyto-percolate.

Another aspect of this invention is directed to a method for treating abnormal insulin release and utilization comprising administering to a mammal (e.g., a female or male human) a therapeutically effective amount of a compound of a phyto-percolate.

In addition to the “direct” effect of the phyto-percolate of this invention there are diseases/conditions wherein subjects with said diseases/conditions will benefit from the associated weight loss and metabolic regulation, such as insulin resistance with impaired glucose tolerance, Type II Diabetes, hypertension, hyperlipidemia, cardiovascular disease, gall stones, certain cancers, sleep apnea, etc. resulting from use of phyto-percolate.

In a further illustrative embodiment a method of making the inventive phyto-percolate is disclosed. The phyto-percolate is prepared by cultivating a mixture of freshwater algae and bacteria that is augmented by a nutrient blend that stimulates the production of fibrinolytic enzymes, forming a fortified algae culture. Added to this fortified algal and bacterial culture is purified fresh water that has been purified by reverse osmosis. The culture is percolated with said purified fresh water and nutrient blend for a predetermined time forming a phyto-percolate that is fibrinolytic in nature. The phyto-percolate is decanted from the fortified algal and bacterial culture and filtered. The filtering of the decanted phyto-percolate is by micro-filtration where the micro-filtration removes particles larger than about 0.22 um.

### BRIEF DESCRIPTION OF THE DRAWINGS

The foregoing and other features and advantages of the present invention will be more fully understood from the following detailed description of illustrative embodiments, taken in conjunction with the accompanying drawing in which:

FIG. 1 is a flow chart showing a method of preparing a phyto-percolate.

### DETAILED DESCRIPTION

This invention will be better understood with resort to the following definitions.

A. phyto-percolate, as used herein refers to the liquid gathered from the algae process described herein.

B. enzymatic extract, as used herein refers to enzymes within the phyto-percolate that break down or lyse surrounding substances including proteins. These enzymes are utilized within the human body to hydrolyze fractured or reduced proteins and soluble pathological fibrin for elimination through the kidneys or liver;

C. proteolytic activity as used herein refers to the process of breaking down or destruction of proteins. The breaking down of proteins, according to the invention, can lead to an increasingly effective immune system;

D. fibrinolytic activity, as used herein refers to the process of breaking down or lysing fibrin. The destruction of soluble fibrin, according to the invention, can lead to an increasingly effective immune system;

E. Therapeutically effective amount, as used herein refers to an amount of phyto-percolate that will alleviate symptoms of a disease state or medical condition generally about 1 or more ounces per day in generally non-symptomatic subjects and about 3 or more ounces per day in diseased subjects; and

F. Enzyme systems, as used herein refers to enzymatic concentrations characterized as responding to an external or internal stimulus.

Detailed embodiments of the present invention are disclosed herein, however, it is to be understood that the disclosed embodiments are merely exemplary of the invention, which may be embodied in various forms. Therefore, specific functional details disclosed herein are not to be interpreted as limiting, but merely as a basis for the claims and as a representative basis for teaching one skilled in the art to variously employ the present invention in virtually any appropriately detailed embodiment.

According to the invention a phyto-percolate is collected from a percolate of a specific group of fresh water algae plants, moss and bacteria. The phyto-percolate collected from such percolate contains an enzymatic extract. The fresh water algae plants and other components of the cultures express enzymes into the surrounding water periodically. These expressed enzymes form the enzymatic extract within the phyto-percolate. These enzymes break down or lyse surrounding proteins either for their own nutrients, to destroy predators, or as a byproduct of other enzymatic pathways. This enzymatic process is known in the art as proteolysis or the expression of a proteolytic agent. Aspects of this enzyme activity are utilized within the human body to hydrolyze fractured or reduced proteins and pathological fibrin for elimination through the kidneys or liver. It is believed that there is a greater abundance of this fractured or reduced protein debris accumulated during chronic illness because of inflammation and ongoing tissue destruction. In many cases, this debris accumulates faster than the human organism is capable of hydrolyzing it for removal, especially with the decreased enzyme production associated with advancing age.

According to the invention, it is believed that the phyto-percolate plays an important role in keeping cells clean and metabolic processes operating effectively. These metabolic processes are believed essential to maintaining a good immune system and proper liver function, aiding in the production of enzymes that are important in preparing and assimilating cellular nutrient in anabolic processes and the repair of injured tissue, and all other bodily functions.

The phyto-percolate contains an enzymatic extract with both proteolytic and fibrinolytic activity. It is believed that this enzymatic extract aids in suppressing the hyper-inflammatory state and normalizing the hyper-coagulate state that is present during chronic illnesses.

It is further believed that use of the phyto-percolate allows tissues to be clean of waste products and metabolic processes to become more efficient and effective, thereby reducing the pathological production of the intra-vascular clots and improving blood circulation throughout the body.

The phyto-percolate dosage will vary with the severity of the disease, the biochemical activity of the disease, and the age and weight of the subject. The effects of using the phyto-percolate will be measured using standard parameters know in the art for any such disease state.

In an adult subject, the dosage will vary from about one ounce per day, generally on an empty stomach, such as for maintenance and the retardation of aging, to about an ounce every hour, up to about 12 ounces per day, in a hospitalized burn or accident case, or during the chemotherapy infusion. The controlled diabetic or cardiovascular subject is generally treated at about two to three ounces of phyto-percolate per day. Dosing on an empty stomach is noted because of the potential for interference on phyto-percolate function from food-stimulated gastrointestinal activities. A 50-70 lb. child is dosed at about three to four ounces per day, generally dosing on an empty stomach, during an acute infection. The greater the free radical oxidative tissue destructive activity caused by age or disease state, the greater the recommended dosage of the phyto-percolate. Without being bound to any particular theory, it is thought that the intake of phyto-percolate per day is more directly related to the severity of oxidative tissue destruction than to the weight of the subject. According to the invention, a phyto-percolate is derived from a culture comprised of freshwater algae, moss and bacteria. Without being bound by any particular theory, it is believed that there are four dominating species of algae in the phyto-percolate culture, four green algae and one moss species. Particular note is made of four green algae species, Stichococcus bacillaris, Stichococcus subtilis, Ulothrix sp. and Chlorella vulgaris. Also, it is believed that one aquatic moss species of the genus Oedocladium is present in the cultures. A deposit of a culture resulting in a phyto-percolate of the present invention has been placed in the American Type Culture Collection, of Manassas, Va., as Deposit #: PTA-5863

Without being bound to any particular theory it is thought that the Stichococcus species and Chlorella vulgaris are the most abundant organisms in each culture and may be the primary enzyme producers.

In particular embodiments an heterotrophic rotifer species exists in the cultures, as well as bacteria that have been identified as Stenotrophomonas maltophilia, Ralstonia pickettii, Ralstonia paucula, Acinetobacter genospecies 11, Acinetobacter junii, Leifsonia aquatica, Riemerella anatipestifer, Variovorax paradoxus, and Streptomyces griseorubens. Without being bound to any particular theory, it is believed that these bacteria may produce proteolytic enzymes that are contributors to the effectiveness of the phyto-percolate.

Phyto-Percolate Production

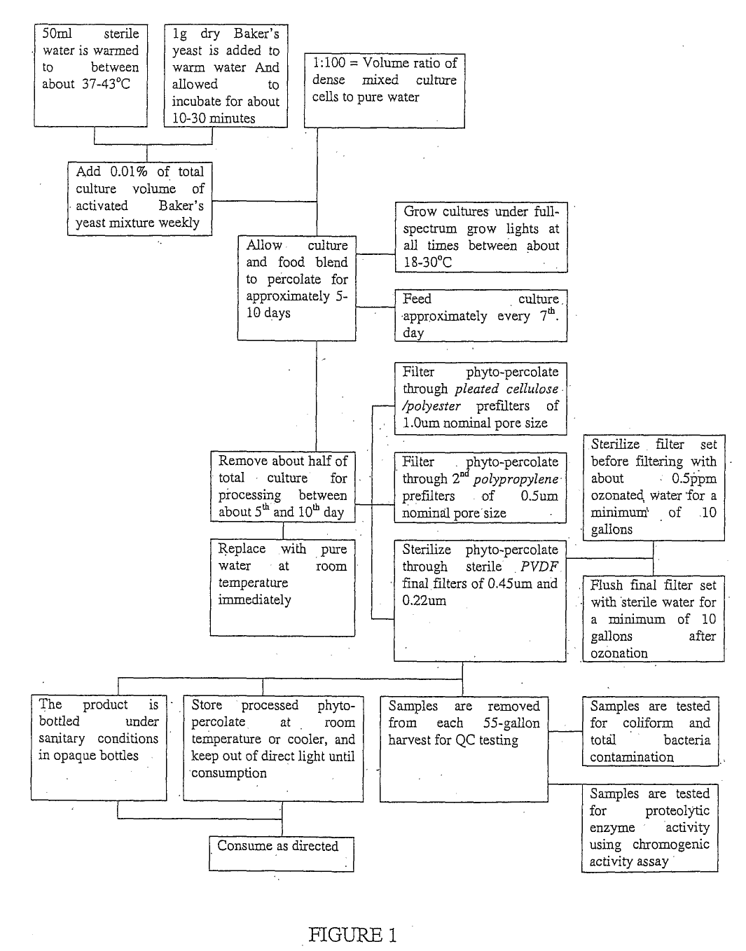

A method of producing phyto-percolate is depicted in FIG. 1. Phyto-percolate cultures of approximately 100-200 ml of dense algal cells in approximately 2.5 gal, or approximately 10 liters, of reverse-osmosis purified sterile water are fed about 1 milliliter (ml) per week of liquid extract of live active yeast, or Baker's yeast, Saccharomyces cerevisiae, which has been prepared from 1.0 g dry active yeast added to 50 ml warm water, at between about 37° and about 43° C. The mixture is allowed to incubate for 10-30 minutes, or until it slightly foams. The cultures are fed in either 1.0 ml weekly doses, or 0.5 ml twice-weekly doses. It is contemplated within the scope of the invention that other yeast cultures may be used. It is further contemplated that other organic nutrients known in the art may be used such as glucose or proteose, or other algal growth media prepared from inorganic nutrients and/or vitamins.

The cultures are grown under full-spectrum grow lights at about 25° C., and produce a final unadjusted pH of between about 6.2 to about 7 that fluctuates over the course of about a week. The cultures are grown in clear glass fishbowl containers having a volume of approximately 2.5 gal with semi-transparent plastic lids, with the exception of about a 3 mm hole in the lid for gas exchange. It is contemplated within the scope of the invention that other culture containers and methods known in the art may be used that allow the algal cells to grow in a layer thin enough for all cells to absorb light. Such methods may include larger batch or continuous type culture systems, and may or may not include aeration.

The cultures are harvested weekly or bi-weekly, between the 5^{rd} and 10^{th} day after feeding, by drawing off the top 1.25 gal of phyto-percolate from each 2.5 gal culture. The algal or other cells and yeast food forming the phyto-percolate culture remain in the bottom of the culture container substantially undisturbed while the phyto-percolate is decanted. The decanted material is then processed as desired. The volume of the container is then optionally returned to original volume. Conveniently this is accomplished with reverse-osmosis purified water at approximately room temperature, about 25° C.

The patterns of harvest and feeding effect enzyme production. Without being bound by any particular theory, it is believed that more frequent smaller feedings such as 0.5 ml twice-weekly may stimulate greater enzyme production than single large amount feedings such as 2 ml bi-weekly, while discouraging bacteria and rotifer colonization. Since enzyme systems are highly dynamic and are directly affected by the immediate surroundings, the suggestion is supported that a food blend such as a liquid extract of active Baker's yeast increases the active proteolytic enzymes in the phyto-percolate culture compared with other foods or nutrient blends.

The peaks of enzyme concentration in the percolate over the course of several weeks are mapped under various feeding regimens, and serve to dictate the optimal date for harvests. According to the invention, the enzyme concentration is analyzed in the cultures and processed phyto-percolate to detect any negative effects of regular harvesting on the algal cultures over time, and is combined with data on the effects of environmental and stress factors such as dark/light, starvation, and/or changes in temperature or pH, which may stimulate or discourage enzyme production. Methods for analyzing these parameters include the isolation and homogenization of select cultures to eliminate all variables besides those being tested, and include constant monitoring of chlorophyll, total protein and enzyme activity on a daily basis or as necessary, utilizing spectro-photometric methods, to measure the health and enzyme activity of the cultures over the course of an isolated-variable experiment.

The method for analyzing proteolytic activity is a typical chromogenic assay using Chromogenix substrate from DiaPharma, S-2251: chromogenic substrate for plasmin and streptokinase-activated plasminogen. Chromogenic substrates are peptides that react with proteolytic enzymes and proportionally change color as the substrate is lysed by the enzymes. The color change can be measured spectro-photometrically over time and is proportional to the proteolytic activity. The chromogenic substrates are made synthetically and are designed to possess a selectivity similar to that of the natural substrate for the enzyme. According to the invention, the substrate for the enzymes present in phyto-percolate are fractionated proteins and fibrin.

Enzyme activity for samples of described phyto-percolate currently ranges from 15-50 mU/mL of plasmin-like activity, when phyto-percolate is prepared as described. These values have been observed clinically to reduce pathological fibrin. Methods for evaluating in vivo effects of phyto-percolate include peripheral blood observations on wet and dry blood smears. Reductions in excess pathological fibrin and platelet aggregation have been observed, which are secondary to inflammation and tissue destruction. Changes in white blood cell mobility have also been observed. Anti-inflammatory effects of phyto-percolate in vivo have also been monitored with independent blood laboratory studies focusing on chronic inflammatory activity and hyper-coagulant states.

Phyto-Percolate Filtration

After harvest of the phyto-percolate from the cultures, the decanted fluid is filtered through a series of depth prefilters and sterile membrane filters made of low-protein binding materials. Examples of suitable final sterilizing filters are provided by Millipore Corp. Durapore brand filters, made of PVDF material. These have been shown to protect the enzyme concentration, and provide a final sterile filtration level of about 0.22 microns, as well as being chemically inert to ozonated water. Ozonated water is used for sterilizing the filter system, as it does not leave a damaging residue like chlorine.

All filters are 10″ cartridge membrane or depth filters of various chemically-inert materials. The prefilters are housed in cartridge filter housings made of styrene-acrylonitrile (SAN). The final filters are housed in polypropylene (PP) housings with Kynar fittings. The material is harvested and filtered using Tygon tubing, peristaltic pumps and 55 gallon containers or other containers that have been pre-sterilized with ozonated water.

The phyto-percolate passes through a filtration regimen comprised of two pre-filters in SAN housings of pore size 1 μm (nominal), made of pleated cellulose/polyester. Examples of these filters are manufactured by Cole-Parmer, Vernon Hills, Ill., USA, catalog number EW-29830-20. It is contemplated within the scope of the invention that other filters know in the art may be used in this step as pre-filters, that are chemically inert.

The phyto-percolate is again filtered using a second stage pre-filter made of polypropylene in a polypropylene housing, with a nominal pore size of about 0.5 um. In one illustrative embodiment, this finishing filter is manufactured by Millipore Corporation, Bedford, Ma., Durapore® brand, Catalog #D00501S01. It is contemplated within the scope of the invention that other filters known in the art may be used in this step as second pre-filters, that are chemically inert.

The phyto-percolate is then passed through a pre-sterilized final filter that sterile-filters the phyto-percolate and removes all traces of bacteria, yeast, mold, algae and other particle contaminants. According to the invention, a final filter set consists of sterile membrane filters in PP housing having progressively smaller pore sizes of 0.45 um and 0.22 μm (absolute). These finishing filters' membranes are made of hydrophilic extremely-low protein-binding PVDF. In one illustrative embodiment, these finishing filters are manufactured by Millipore Corporation, Durapore® brand, Catalog #'s CVHI01TPE and CVDI01TPE. It is contemplated within the scope of the invention that other filters know in the art may be used that are chemically inert and can be sterilized using 0.5 ppm ozonated water for a flush of no less than 10 gallons, followed by a flush of no less than 10 gallons of sterile water, before filtering the phyto-percolate.

Filtration by size exclusion removes approximately >99.9% of contaminants such as bacteria, yeast and mold spores, and algal cells. It is also believed to preserve enzymatic activity if filter materials are made of low-protein-binding, chemically-inert materials. The resulting liquid, the phyto-percolate, is substantially comprised of water, active enzymes and sugars. The phyto-percolate, after passing through the finishing filter is then usefully stored in sealed sterile 55 gal HDPE drums at between 21° and 27° C. until bottling. Samples are taken from each batch immediately after filtering to test for enzyme efficacy and contamination and for standardization. The acceptable values for fibrinolytic enzyme efficacy in the human body are observed in the phyto-percolate as between 15 and 50 milli-units of plasmin-like activity.

The phyto-percolate is processed and bottled under sanitary conditions known in the art using ozone sterilization. It is believed that this step avoids enzyme degradation associated with the use of chlorine or heat sterilization because ozone leaves no residue if left to dissipate for a period of 24 hours, or if followed by a rinse of sterile water It is contemplated within the scope of the invention that other methods of filtration and sterilization known in the art may be used that are not unreasonably degrading of the enzymatic activity. The phyto-percolate is usefully packaged in opaque UV-protected bottles and shipped with cold packs to reduce product degradation.

### EXAMPLE 1 TREATMENT OF HIV

A twenty-three year old male presents with an elevated T-cell count and a determinable viral load of HIV virus. The subject is administered one ounce per day of phyto-percolate for seven days. The subject is tested thirty days thereafter and is found to have a substantially reduced or sub-determinable viral load.

### EXAMPLE 2 TREATMENT OF ACNE

An 18 year old female presents with mild to moderate cystic acne. The subject is administered one ounce twice a day of phyto-percolate for 30 days. Acne is substantially reduced after three weeks and such reduction continues over the course of treatment.

### EXAMPLE 3 TREATMENT DURING CHEMOTHERAPY

A 54 year old subject is undergoing doxorubicin therapy for treatment of cancer. On days when the chemotherapeutic agent is administered, the subject ingests 1 oz of phyto-percolate per hour for 8 hours. The side effects of chemotherapy are thereby significantly relieved.

Although, the use of phyto-percolate in the above examples are upon human subjects, it should be understood by those skilled in the art that the phyto-percolate may be used in animals to treat similar disease states (e.g arthritis, cancer, cardio-vascular disease, etc.). Likewise it should be understood that the phyto-percolate can be used to enhance the well being and performance of animals.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

The foregoing has been a description of an illustrative embodiment of the present invention. While several illustrative details have been set forth, such are only for the purpose of explaining the present invention. Various other changes, omissions and additions in the form and detail thereof may be made therein without departing from the spirit and scope of the invention.

What is claimed is:

1. A method of treating or preventing a disease or disorder in a human that is capable of being treated or prevented by phyto-percolate, said method comprising administering to human in need of such treatment or prevention a therapeutically effective amount of phyto-percolate.

2. The method according to claim 1 wherein said disease is diabetes and said therapeutically effective amount is about 1 ounce to about 12 ounces in divided daily doses of 1-2 oz. of said phyto-percolate.

3. The method according to claim 1 wherein said disease is HIV and said therapeutically effective amount is about 1 ounce to about 12 ounces in divided daily doses of 1-2 oz. of said phyto-percolate.

4. The method according to claim 1 wherein said disease is cystic acne and said therapeutically effective amount is about 1 ounce to about 12 ounces in divided daily doses of 1-2 oz. of said phyto-percolate.

5. The method according to claim 1 wherein said disease is high blood pressure and said therapeutically effective amount is about 1 ounce to about 12 ounces in divided daily doses of 1-2 oz. of said phyto-percolate.

6. The method according to claim 1 wherein said disease is cancer and said therapeutically effective amount is about 1 ounce per hour of said phyto-percolate during chemotherapy treatment.

7. The method of claim 1 wherein said disease or disorder is selected from the group consisting of cancer, diabetes, arthritis, cardio-vascular disease, cerebral vascular disease peripheral vascular disease, compromised immune system disorders, HIV/AIDS, and metabolic disorders.

8. The method of claim 1 wherein said disease or disorder is a compromised immune system caused by chemotherapy.

9. The method of claim 7 wherein said compromised immune system and compromised liver function are caused by the HIV virus or by anti-viral therapies.

10. The method of claim 9 wherein said therapeutically effective amount of phyto-percolate is administered in conjugation with protease inhibitors, specifically protease inhibitors that block the replication of HIV.

11. The method of claim 9 wherein said therapeutically effective dosage is from about 1 ounce to about 12 ounces per day.

12. The method of claim 11 wherein said therapeutically effective dosage is from about 2 ounces to 4 ounces a day.

13. A method of producing a phyto-percolate comprising the steps of:
(i) providing a fresh water algal and bacterial culture of ATCC Deposit #: PTA-5863
(ii) growing said algal and bacterial culture in purified water;
(iii) feeding said culture a nutrient blend to produce phyto-percolate;
(iv) decanting said phyto-percolate from said culture; and
(v) filtering said decanted phyto-percolate.

14. The method according to claim 13, wherein said filtering step is by size exclusion.

15. The method according to claim 13, wherein said filtering step preserves enzymatic activity of said phyto-percolate.

16. The method according to claim 13, wherein said fresh water algal and bacterial culture contains at least four said green algae, one said moss species and nine said listed bacterial species.

17. The method according to claim 13, wherein said nutrient blend is a liquid extract of live Saccharomyces cerevisiae.

18. The method according to claim 17, wherein said live Saccharomyces cerevisiae is introduced into said cultures as a means of stimulating enzymatic production by said algal and bacterial culture.
